(19) United States
(12) Patent Application Publication
(10) Pub. No.: US 2020/0390924 A1
(43) Pub. Date: Dec. 17, 2020

(54) DEODORIZER

(71) Applicant: Ushio Denki Kabushiki Kaisha, Tokyo (JP)

(72) Inventor: Keisuke NAITO, Tokyo (JP)

(73) Assignee: Ushio Denki Kabushiki Kaisha, Tokyo (JP)

(21) Appl. No.: 16/957,040

(22) PCT Filed: Oct. 22, 2018
(86) PCT No.: PCT/JP2018/039194

§ 371 Date: Jun. 22, 2020

(30) Foreign Application Priority Data
Dec. 26, 2017 (JP) 2017-249041

(51) Int. Cl. A61L 9/015; A61L 9/20

(52) U.S. Cl. CPC A61L 9/015; A61L 2209/212; A61L 2209/134; A61L 9/20

(57) ABSTRACT

[Problem] To provide a deodorizer that supplies ozone-containing gas to a spot to be deodorized and deodorizes same and that can break down and remove odoriferous substances existing inside an object to be deodorized such as a textile product.

[Solution] Provided is a deodorizer which includes a deodorizing bed that can be pressed against a spot to be deodorized, and is characterized in that the deodorizing bed has: a first room which has an air supply port to supply ozone-containing gas to the spot to be deodorized; a second room which is provided adjacent to the first room and has an exhaust port that suctions the gas supplied to the spot to be deodorized; and a partition that separates the first room from the second room.

### TECHNICAL FIELD

The present invention relates to a deodorizer for textile products, and more particularly to a deodorizer that decomposes odorous substances using ozone.

### BACKGROUND ART

Odorous substances readily adhere to textile products such as carpets, or seats in theaters, movie houses, or vehicles and of home sofas and the like. Since the seats in movie houses or vehicles, for example, are not removable, deodorization is performed by spraying a deodorizer or the like. This, however, deodorizes only the surfaces of the seats and is hardly effective in deodorizing the inside of the seats.

Air purifiers that remove volatile organic compounds (VOC) contained in the floor or walls have been known. JP-A-2007-152026 (Patent Document 1), for example, discloses a structure of an air purifier, which is illustrated in FIG. 8.

Referring to FIG. 8, the air purifier 20 includes a cleaning head 21. Air heated by a heater part (not shown) is delivered through an air blow passage 22 to an air blow chamber 24, and blown to a floor surface or the like from an outlet 26 at the lower end thereof to cause the volatile gas (VOC gas) contained in the building material to volatilize.

The volatilized VOC gas is sucked into a suction chamber 25 from a suction port 27, where the gas is oxidized and decomposed by ozone from an ozone generator 30. The air after the VOC gas has been removed therefrom is exhausted to the outside through a suction passage 23.

The device according to Patent Document 1 is therefore one that sucks the VOC gas contained in building materials and removes this sucked VOC gas inside the cleaning head by decomposition using ozone, rather than one that removes odorous substances contained inside textile products such as seats by decomposition.

Namely, the device merely performs decomposition of the VOC gas sucked out of an object such as a building material using ozone.

If this air purifier were used for deodorization of unwashable textile products such as carpets and seats, the ozone would be used only for the decomposition of a VOC gas sucked out of the textile product. Namely, the issue here is that the odorous components remaining inside the object to be deodorized cannot be removed by decomposition since the ozone is not supplied to the inside of the textile product.

### PRIOR ART DOCUMENT

### Patent Document

Patent Document 1: JP-A-2007-152026

### SUMMARY OF THE INVENTION

### Problems to be Solved by the Invention

In view of the issue in the prior art as described above, an object of this invention is to provide a deodorizer that supplies a gas containing ozone to a spot to be deodorized to deodorize the spot, the deodorizer being capable of removing odorous substances contained inside an object to be deodorized such as a textile product by decomposition.

### Means for Solving the Problems

To solve the problem described above, the deodorizer according to this invention includes a deodorizing head abutted to a spot to be deodorized, and the deodorizing head includes a first chamber provided with an intake port allowing a gas containing ozone to be supplied to the spot to be deodorized, a second chamber provided adjacent the first chamber and including an exhaust port allowing a gas that has been supplied to the spot to be deodorized to be sucked, and a partition that separates the first chamber from the second chamber.

In the deodorizer, the partition extends as far as to a lower end of the deodorizing head.

The deodorizer further includes an ultraviolet lamp that radiates ultraviolet of wavelengths of 200 nm or less provided in the first chamber.

The deodorizer further includes an ozone decomposer provided in the second chamber.

The deodorizer further includes an ultraviolet lamp that radiates light of wavelengths of 230 to 300 nm disposed inside the second chamber.

### Effect of the Invention

The deodorizer according to this invention supplies a gas containing ozone to a spot to be deodorized of an object to be deodorized such as a textile product from an intake port of a first chamber to deliver the ozone as far as to the inside of the object to be deodorized so that odorous substances contained inside the object to be deodorized are effectively decomposed and removed by the ozone.

### BRIEF DESCRIPTION OF THE DRAWINGS

FIG. 1 is a cross-sectional side view of a first embodiment of the present invention.

FIG. 2 is a cross-sectional side view of a second embodiment of the present invention.

FIG. 3 is a cross-sectional side view of a third embodiment of the present invention.

FIG. 4 is a cross-sectional side view of a fourth embodiment of the present invention.

FIG. 5 is a cross-sectional side view of a fifth embodiment of the present invention.

FIG. 6 is a cross-sectional side view of a sixth embodiment of the present invention.

FIG. 7 is a table that shows the effects of the present invention.

FIG. 8 is a cross-sectional view of an existing technique.

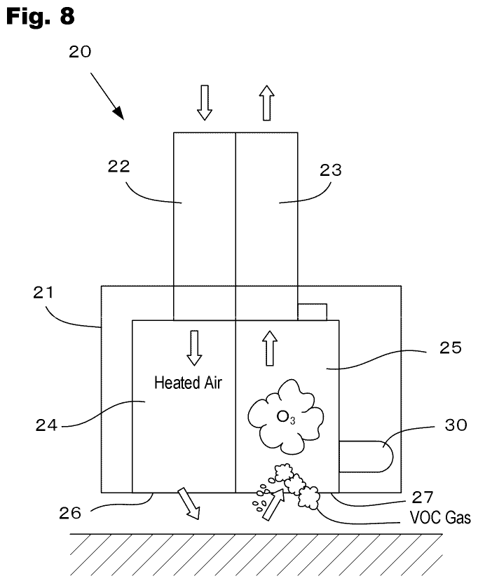

### MODE FOR CARRYING OUT THE INVENTION

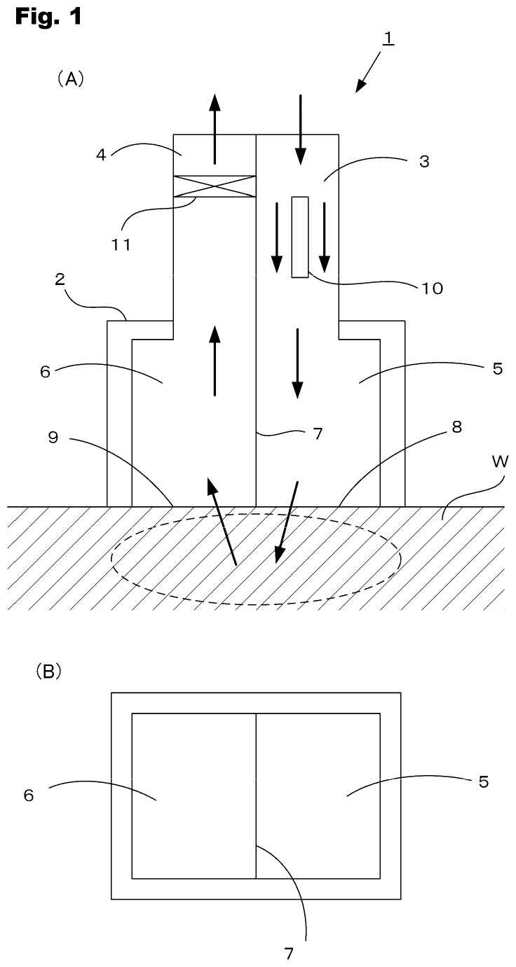

FIG. 1 illustrates a deodorizer 1 that uses ozone according to the present invention. This deodorizer 1 includes a deodorizing head 2 at the lower end, and a pair of an intake passage 3 and an exhaust passage 4. The lower ends of these intake passage 3 and exhaust passage 4 are connected respectively to a first chamber 5 and a second chamber 6.

As illustrated also in FIG. 1(B), the first chamber 5 and second chamber 6 are separated by a partition 7, with an intake port 8 formed at the lower end of the first chamber 5 and an exhaust port 9 formed at the lower end of the second chamber 6. The partition 7 extends as far as to the lower end of the deodorizing head 2 so that, when the deodorizing head 2 is abutted to a spot to be deodorized of an object to be deodorized W such as a seat or carpet, the partition 7 also makes contact with the spot to be deodorized of the object to be deodorized W.

An ultraviolet lamp 10 is provided as an ozone supplier in the intake passage 3. This ultraviolet lamp 10 radiates ultraviolet (vacuum ultraviolet) of wavelengths of 200 nm or less. A xenon excimer lamp is adopted as one example thereof.

An ozone decomposer 11 is provided in the exhaust passage 4. This ozone decomposer 11 uses an ozone decomposing catalyst (filter), activated carbon, a heat source such as a halogen heater, and an ultraviolet light source (such as a lamp or LED) that radiates light of wavelengths of 230 to 300 nm.

As illustrated in FIG. 1, the deodorizing head 2 of the deodorizer 1 is abutted to a spot to be deodorized of an object to be deodorized W, such as a seat or carpet. When air is supplied from the intake passage 3, ozone is generated from the oxygen in the air by the vacuum ultraviolet radiation from the ultraviolet lamp 10. This ozone and air are blown to the object to be deodorized W through the first chamber 5 and from the intake port 8 at the end thereof, reaching the inside of the object, where the ozone reacts with and decomposes the odorous substances.

The air is then sucked from the exhaust port 9 into the second chamber 6, and discharged to the outside through the exhaust passage 4. The ozone decomposer 11 in the exhaust passage 4 decomposes and removes the remaining ozone as the air is discharged. This prevents any unpleasantness that may be felt by the operator.

Since the partition 7 separating the first chamber 5 from the second chamber 6 extends as far as to the lower end of the deodorizing head 2, the partition 7 also contacts the object to be deodorized W, which prevents the air and ozone from the intake port 8 of the first chamber 5 from bypassing and flowing into the exhaust port 9 of the second chamber 6, and allows the air and ozone to precisely reach the inside of the object to be deodorized W.

The odorous substances left inside the object to be deodorized W are thus reliably decomposed and removed.

While the partition 7 has been described above as extending as far as to the lower end of the deodorizing head 2 to contact the object to be deodorized W, strictly speaking, the lower ends of the deodorizing head 2 and the partition 7 need not necessarily be completely flush with each other. The partition 7 need only extend to a point where it can prevent the air and ozone supplied from the intake port 8 of the first chamber 5 from bypassing and flowing into the exhaust port 9 of the second chamber 6 and failing to reach the inside of the object to be deodorized W.

While the configuration described above includes a blower fan in the intake passage 3 and a suction fan in the exhaust passage 4, the suction fan alone may suffice depending on the flow rate or other conditions.

The intake passage 3 and first chamber 5 are separate structures, and the exhaust passage 4 and second chamber 6 are separate structures in the embodiment described above. In an alternative configuration they may each be united, i.e., the intake passage 3 and the first chamber 5 may be united, and the exhaust passage 4 and the second chamber 6 may be united.

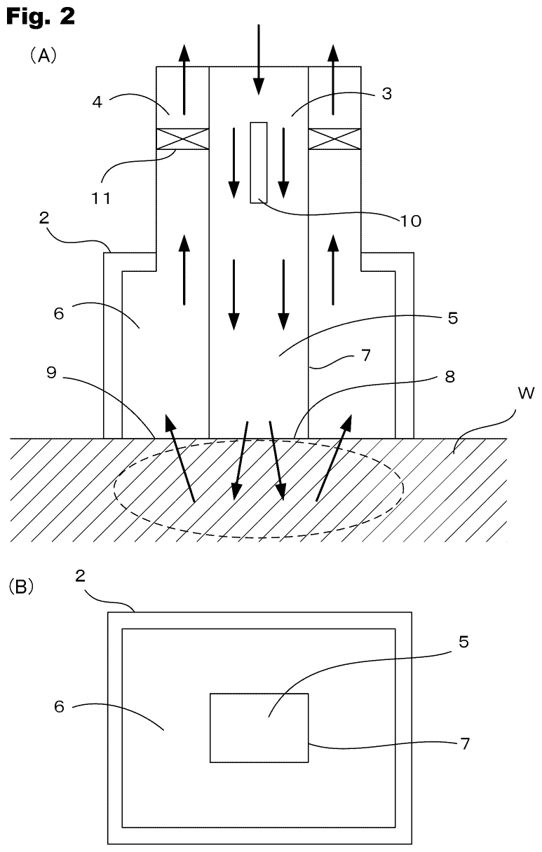

FIG. 2 illustrates a second embodiment with a different deodorizing head 2. As illustrated in FIG. 2(B), the first chamber 5 and second chamber 6 are disposed concentrically. Namely, the second chamber 6 is formed around the circumference of the first chamber 5. While the first chamber 5 and second chamber 6 have a rectangular cross section as illustrated in FIG. 2(B), they may have a concentric circular section, or a concentric oval section.

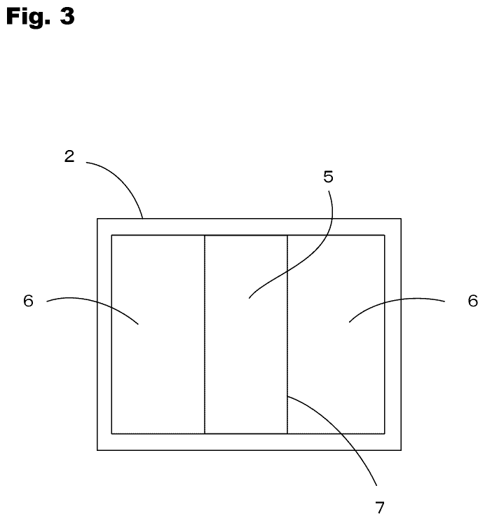

Moreover, the first chamber 5 and second chamber 6 may not be concentric to each other, as illustrated in FIG. 3, with second chambers 6 disposed on both sides of the first chamber 5 in the middle.

In this second embodiment, the air and ozone are supplied from the first chamber 5 in the middle and reach the inside of the object to be deodorized W, and, after decomposing and removing odorous substances there, are exhausted through the second chambers 6 on the outer sides.

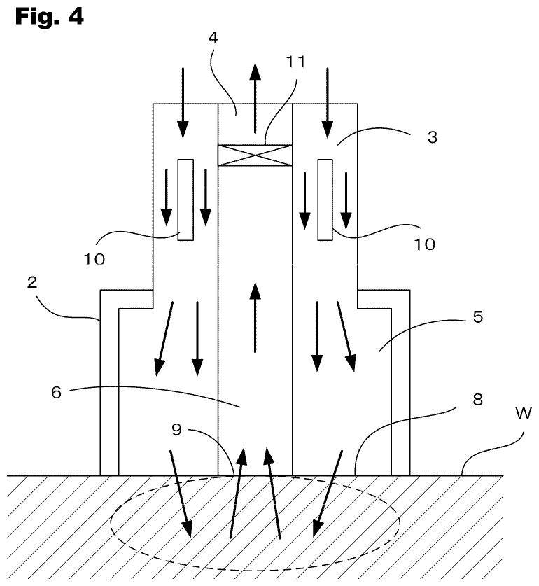

FIG. 4 illustrates a fourth embodiment. In this embodiment, the relationship between the first chamber 5 and second chamber 6 in the second embodiment of FIG. 2 are inverted, i.e., the second chamber 6 is disposed in the middle, and the first chamber 5 is disposed therearound.

In this case, too, an ultraviolet lamp 10 is disposed in the first chamber 5, and an ozone decomposer 11 is disposed in the second chamber 6.

In this fourth embodiment, ozone is generated as the air flowing into the surrounding first chamber 5 is exposed to the ultraviolet from the ultraviolet lamp 10. This ozone and the air flowing into the object to be deodorized W decompose and remove counterflowing odorous substances. The air after the deodorization process is sucked into the second chamber 6 in the middle, where the remaining ozone is decomposed and removed by the ozone decomposer 11, and discharged to the outside through the exhaust passage 4.

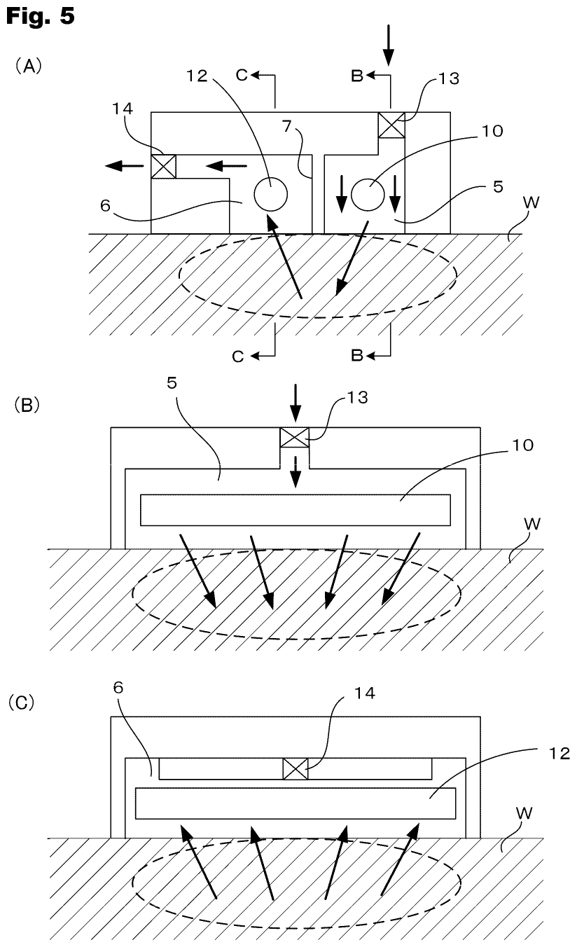

FIG. 5 illustrates yet another or fifth embodiment, wherein (A) is a cross-sectional side view, (B) is a cross-sectional view along B-B in (A), and (C) is a cross-sectional view along C-C.

In this embodiment, an ultraviolet lamp 10 (vacuum ultraviolet lamp) that radiates ultraviolet (vacuum ultraviolet) of wavelengths of 200 nm or less, and an ultraviolet lamp 12 that radiates light of wavelengths of, for example, 230 to 300 nm, are disposed inside the first chamber 5 and inside the second chamber 6, respectively, which are separated by the partition 7.

For this ultraviolet lamp 12, a phosphor lamp with an arc tube of a xenon excimer lamp coated with a phosphor that is excitable by vacuum ultraviolet may be used. Examples of phosphors that emit light of this wavelength range include praseodymium-activated lanthanum phosphate, praseodymium-activated yttrium aluminoborate, and the like.

A blower fan 13 and a suction fan 14 are respectively provided in the intake passage 3 connected to the first chamber 5 and in the exhaust passage 4 connected to the second chamber 6.

The air delivered by the blower fan 13 through the intake passage 3 into the first chamber 5 generates ozone by the vacuum ultraviolet radiation from the vacuum ultraviolet lamp 10, and this ozone is delivered into the object to be deodorized W to decompose the odorous substances. The spot to be deodorized is directly irradiated with the ultraviolet from the ultraviolet lamp 12 inside the second chamber 6 so that the odorous substances present in this spot to be deodorized are decomposed by the action of light.

At this time, the ozone generated inside the first chamber 5 and reaching the spot to be deodorized absorbs the light from the ultraviolet lamp 12 and produces active oxygen species and radicals, which also contribute to deodorization of the object to be deodorized W, resulting in an increased deodorization effect.

The gas that worked for the deodorization in this way is then exhausted to the outside through the exhaust passage 4 by the suction fan 14.

While a vacuum ultraviolet lamp is used as the ozone generator in the first chamber in the embodiments described above, the ozone generator is not limited to this and may use plasma discharge or dielectric barrier discharge.

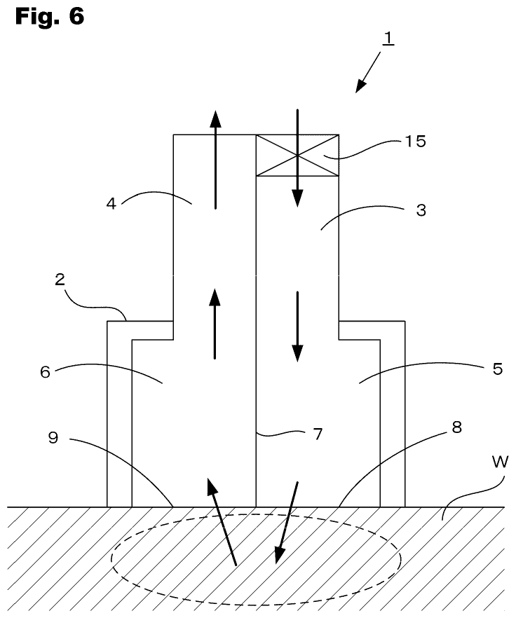

FIG. 6 illustrates a deodorizer that includes an ozone generator other than a vacuum ultraviolet lamp. An ozone generator 13 that uses plasma discharge or dielectric barrier discharge is provided in the intake passage 3 of the first chamber 5.

The ozone generator 13 generates ozone from the air flowing into the intake passage 3, which is then supplied to the object to be deodorized W from the intake port 8 of the first chamber 5.

Experiments were conducted to verify the effects of the present invention.

Nine 7 cm×7 cm pieces of carpets were prepared as samples, which were divided into 3 groups. Solutions of the following 3 types of malodorous substances were dropped on the three carpets of each group:

(1) A 10 μL solution of trans-2-Nonenal (Wako 1st Grade, produced by Wako Pure Chemical Industries, Ltd.) was dropped on the three carpets of the first group.

(2) A 10 μL solution of isovaleric acid (Wako Special Grade, produced by Wako Pure Chemical Industries, Ltd.) diluted at 1:1000 by pure water was dropped on the three carpets of the second group.

(3) Ammonia water (25%, 10 μL) (Wako 1st Grade, produced by Wako Pure Chemical Industries, Ltd.) was dropped on the three carpets of the third group.

The following three treatments were carried out to each one of each group of the three types of samples:

(1) Treatment using the deodorizer according to the first embodiment of the present invention for 30 seconds.

(2) Suction only for 30 seconds, with the vacuum ultraviolet lamp in the first embodiment being turned off (Comparative Example 1).

(3) Left without any treatment (Comparative Example 2).

### <Odor Evaluation Method and Results>

Sensory evaluation was conducted by five panels. Specifically, after performing the three types of treatments listed above to each of the samples of three types of malodorous substances, the samples were ranked in the order of the intensity of the odor perceived when smelled close to the spot where the sample solutions were dropped, after the treatment.

The odor indexes are the points scored in accordance with the following 6-scale odor intensity index method.

### <Odor Intensity Evaluation Standards (6-Scale Odor Intensity Index Method)>

5 points: Very strong smell

4 points: Strong smell

3 points: Easily perceptible smell

2 points: Weak but recognizable smell

1 point: Barely perceptible smell

0 point: No smell

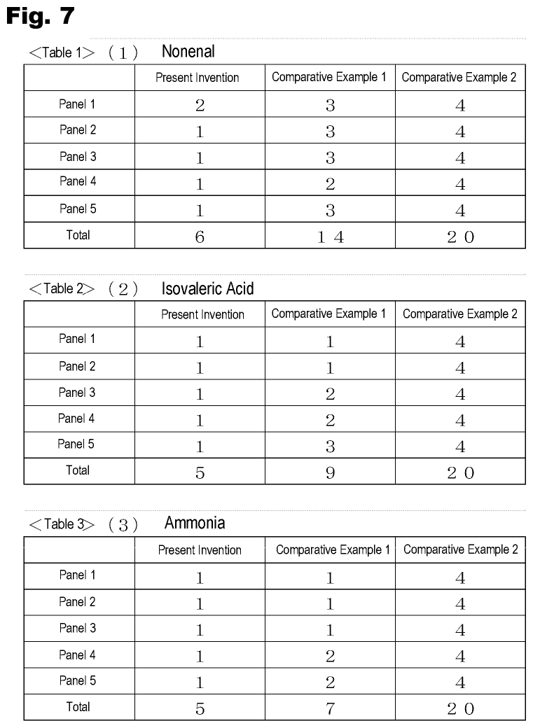

The results are shown in Tables 1 to 3 in FIG. 7. The effects of the present invention were validated in each case.

As has been described above, the deodorizer according to the present invention supplies ozone to the inside parts of textile products such as seats and carpets to be deodorized, so that odorous substances that have infiltrated into these parts can be effectively removed by decomposition using the ozone.

The ozone that has been supplied from the first chamber to the object to be deodorized, after having reached the inside of the object to be deodorized, moves to the second chamber. The ozone is thus concentrated to the spot to be deodorized so that an efficient deodorization effect is achieved without the need to increase the ozone concentration in other regions. The partition separating the first chamber from the second chamber extending as far as to contact the spot to be deodorized prevents the ozone from the first chamber from bypassing and flowing into the second chamber, and allows the ozone to reach deep into the spot to be deodorized to effectively decompose and remove the odorous substances.

### DESCRIPTION OF REFERENCE SIGNS

1: Deodorizer
2: Deodorizing head
3: Intake passage
4: Exhaust passage
5: First chamber
6: Second chamber
7: Partition
8: Intake port
9: Exhaust port
10: (Vacuum) ultraviolet lamp
11: Ozone decomposer
12: Ultraviolet lamp
13: Blower fan
14: Suction fan
15: Ozone generator
W: Object to be deodorized

1. A deodorizer that supplies a gas containing ozone to a spot to be deodorized and deodorizes the spot, comprising
a deodorizing head abutted to a spot to be deodorized,
the deodorizing head including a first chamber provided with an intake port allowing a gas containing ozone to be supplied to the spot to be deodorized,
a second chamber provided adjacent the first chamber and including an exhaust port allowing a gas that has been supplied to the spot to be deodorized to be sucked, and
a partition that separates the first chamber from the second chamber.

2. The deodorizer according to claim 1, wherein the partition extends as far as to a lower end of the deodorizing head.

3. The deodorizer according to claim 1, further comprising an ultraviolet lamp that radiates ultraviolet of wavelengths of 200 nm or less provided in the first chamber.

4. The deodorizer according to claim 1, further comprising an ozone decomposer provided in the second chamber.

5. The deodorizer according to claim 1, further comprising an ultraviolet lamp that radiates light of wavelengths of 230 to 300 nm disposed inside the second chamber.
